(19) United States
(12) Patent Application Publication
(10) Pub. No.: US 2026/0133198 A1
(43) Pub. Date: May 14, 2026

(54) DIAGNOSIS AND TREATMENT OF DISEASES COMPLICATED BY SOLUBLE TARGET IN BLOOD

(71) Applicant: Curadel Surgical Innovations, Inc., Natick, MA (US)

(72) Inventors: John V. Frangioni, Weston, MA (US); John Mark Brimacombe, Charlottesville, VA (US)

(73) Assignee: Curadel Surgical Innovations, Inc., Natick, MA (US)

(21) Appl. No.: 19/385,717

(22) Filed: Nov. 11, 2025

Related U.S. Application Data
(60) Provisional application No. 63/720,039, filed on Nov. 13, 2024.

(51) Int. Cl. G01N 33/573; G01N 1/40

(52) U.S. Cl. CPC G01N 33/573; G01N 1/405; G01N 2333/948; G01N 2800/52

(57) ABSTRACT

In accordance with at least one aspect of this disclosure, a method for preparing a patient for a diagnostic procedure and/or for receiving a therapy using a disease targeted drug is provided. The method can include the steps of a) identifying one or more soluble targets detectable in plasma of the patient; and b) extracorporeally removing the soluble targets from the plasma of the patient. Extracorporeally removing the soluble targets from the plasma of the patient can include using plasmapheresis or immunoadsorption to prepare a patient for a procedure using disease targeted treatment or diagnostics. Additionally, or alternatively, dose modulation can be used to proportionally increase the concertation of the diagnostic/treatment agent relative to the concentration of the soluble target in the blood, improving the outcome treatment and diagnostic procedures over conventional methods.

### CROSS-REFERENCE TO RELATED APPLICATIONS

This application claims claims priority to and the benefit of U.S. Provisional Patent Application No. 63/720,039, filed Nov. 13, 2024, the entire content of which is incorporated herein by reference.

### FIELD OF THE INVENTION

The present invention relates to methods and strategies for improved diagnosis and treatment of diseases complicated by soluble target in blood. Additional methods can include methods for preparing a patient for diagnosis and treatment of diseases complicated by soluble target in blood.

### BACKGROUND

Many diseases overexpress one or more proteins that can be used as the target for diagnostic and treatment drugs. When the target protein remains at the site of disease, detection and treatment using target-specific ligands is typically effective. However, if the ligand binding portion of the protein is also present in blood (i.e., plasma) due to proteolytic cleavage, expression of a soluble form, etc., then binding to the site of disease becomes difficult because injected drug is adsorbed to the soluble form in blood. Such soluble targets floating in plasma acts as a sink that will sequester an injected drug and change its pharmacokinetics and clearance. In many cases, the concentration of soluble target in plasma is high enough that it creates an unfavorable gradient between the blood and the target site. In certain instances, the soluble target in plasma can be too large to be filtered by the kidney, thus in such cases, it is not possible to extract the drug from plasma and eliminate it into urine. In certain cases, it may also be possible that a plasma protein-bound ligand will be recognized by the liver and cleared hepatically.

Certain conventional techniques, such as blocking a soluble target in plasma with cold drug, either by giving extra cold drug or injecting hot drug of low specific activity (which has a high fraction of cold drug in it) only serve to lower the total amount of drug reaching the site of disease. In the case of diagnostic and therapeutic drugs that use radioactivity, these conventional techniques may lower the number of hot (radioactive) atoms bound to plasma, the number of hot atoms bound to the site of disease is proportionally lowered as well, achieving no net improvement.

Small zwitterionic molecule anti-cancer drugs having more than one targeting ligand binding to at least one receptor on tumor cells represent new and improved anti-cancer drugs greatly improving performance in both diagnosing and treating malignant tumors. A key feature of zwitterionic drugs is shifting of clearance, i.e., elimination from the body, from mostly hepatic (liver) to mostly or exclusive renal (kidney). Drugs exhibiting renal clearance are eliminated from the body in urine.

Accordingly, there remains a need in the art for methods for preparing a patient for diagnosis and treatment of diseases complicated by soluble target in blood, for example, by lowering or eliminating the amount of soluble target in the plasma before diagnostic procedures and/or before treatment, or by manipulating the dosing schedule to account for changes of soluble target in blood over time.

### SUMMARY

In accordance with at least one aspect of this disclosure, a method for preparing a patient for a diagnostic procedure and/or for receiving a therapy using a disease targeted drug is provided. The method includes the steps of: a) identifying one or more soluble targets detectable in plasma of the patient; and b) extracorporeally removing the soluble targets from the plasma of the patient.

In certain embodiments, extracorporeally removing the soluble targets from the plasma of the patient in step b) can include performing plasmapheresis. In certain such embodiments, performing plasmapheresis can further include c) performing plasma exchange, including removing blood from the patient and separating plasma from the blood, discarding the plasma, and providing new plasma to the patient to non-selectively remove the one or more soluble targets within the plasma of the patient. The soluble target can be fibroblast activation protein (FAP) and/or prostate-specific membrane antigen (PMSA).

In certain embodiments, extracorporeally removing the soluble targets from the plasma of the patient in step b) can include extracorporeally removing the one or more soluble targets from the plasma of the patient via immunoadsorption, and immunoadsorption can further include: c) removing blood from the patient and separating plasma from the blood, d) passing the plasma through an immunoadsorption column to selectively remove the soluble target identified in step a) from the plasma, and e) returning the treated plasma to the patient.

The immunoadsorption column can include a filter medium having one or more targeting ligands bound thereto, for example one or more targeting ligands configured to bind FAP or PMSA, where the one or more soluble targets includes FAP and/or PMSA.

In certain embodiments, the method can further include the steps of: d) administering a first dose of a disease targeted diagnostic agent or treatment agent to the patient at a first concentration, wherein the disease targeted diagnostic agent or treatment agent is targeted at a biological abnormality caused by a disease overexpressing the one or more soluble targets such that the first dose lowers an initial concertation of the one or more soluble targets in the plasma of the patient, and e) administering a subsequent dose of the disease targeted diagnostic agent or treatment agent to the patient at a second concentration higher than the first concentration, wherein the second concentration is determined as a function of a decrease in the initial concentration of the one or more soluble targets in the plasma.

In certain embodiments, the method can further include the steps of: d) administering a first dose of a disease targeted diagnostic agent or treatment agent to the patient at a first concentration, wherein the disease targeted diagnostic agent or treatment agent is targeted at a biological abnormality caused by a disease overexpressing the one or more soluble targets such that the first dose lowers an initial concertation of the one or more soluble targets in the plasma of the patient; and e) administering additional subsequent doses of the disease targeted diagnostic agent or treatment agent to the patient at a respective subsequent concentration that increases with each administration at a rate proportional to a decrease of the concentration of the one or more soluble targets in the plasma of the patient.

In accordance with at least one aspect of this disclosure, a method of improving diagnosis and treatment of diseases complicated by one or more soluble targets in blood is provided. The method can include the steps of: a) preparing a patient using any one or more of the preparation methods shown and described herein, and b) administering a disease targeted diagnostic agent or treatment agent to the patient at a higher dose than if the patient had not been prepared using one or more of the preparation methods described herein. In certain embodiments, the steps a) and b) are performed sequentially.

In certain embodiments, the step b) can further include administering the disease targeted diagnostic agent or treatment agent to the patient such that at least 80% of an injected dose of the disease targeted diagnostic agent or treatment agent not binding the target is passed through urine within 48 hours of administration, or where at least 90% of the injected dose of the disease targeted diagnostic agent or treatment agent not binding the target is passed through urine within 48 hours of administration, or where at least 80% of the injected dose of the disease targeted diagnostic agent or treatment agent not binding the target is passed through urine within 24 hours of administration, or where at least 90% of the injected dose of the disease targeted diagnostic agent or treatment agent not binding the target is passed through urine within 24 hours of administration.

In accordance with at least one aspect of this disclosure, a method of improving diagnosis and treatment of diseases complicated by soluble target in blood is provided, the method including the steps of (which in certain embodiments are performed sequentially): a) preparing a patient using any one or more of the preparation methods described herein, and b) administering a disease targeted diagnostic agent or treatment agent to the patient such that a prescribed concentration of an injected dose of the disease targeted diagnostic agent or treatment agent not binding the target is removed from blood faster and is passed through urine within a prescribed time after administration. In certain such embodiments, the prescribed concentration is greater than if the patient had not been prepared by any of the preparation methods described herein, and where the prescribed time after administration is less than if the patient had not been prepared by any of the preparation methods described herein.

In accordance with at least one aspect of this disclosure, a method of improving diagnosis and treatment of diseases complicated by soluble target in blood is provided, the method including the steps of: a) administering a first dose of a disease targeted diagnostic agent or treatment agent to the patient at a first concentration, wherein the disease targeted diagnostic agent or treatment agent is targeted at a biological abnormality caused by a disease overexpressing the soluble target such that the first dose lowers an initial concertation of the soluble target in the plasma of the patient; and b) administering additional subsequent doses of the disease targeted diagnostic agent or treatment agent to the patient at a respective subsequent concentration that increases with each administration at a rate proportional to the decrease of the concentration of the soluble target in the plasma of the patient.

In certain embodiments, administering additional subsequent doses of the step b) can include administering the additional subsequent doses at a maximum concentration without overdosing off-target, non-diseased tissues and organs.

In certain embodiments, the disease targeted diagnostic agent or treatment agent can be or include a small-zwitterionic molecule having a core chemical structure located at a center and two or more substituents for binding zwitterionic linkers and a targeting ligand bound to the core.

In certain embodiments, the soluble target is or includes fibroblast activation protein (FAP) and/or prostate-specific membrane antigen (PMSA), and the targeting ligand is a molecule that binds FAP or PMSA.

In certain embodiments, administering additional subsequent doses in the step b) can include administering the additional subsequent doses such that at least 80% to 90% of an injected dose of the disease targeted diagnostic agent or treatment agent not binding the target is passed through urine within 24 to 48 hours of administration.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 illustrates blood and urine curves over time, where the left axis and red curve show plasma concentration over time and the right axis and blue curve show percent of injected dose in urine;

FIGS. 2a and 2b show the results of a simulation with a cell count (3×10⁸) with no internalization for the total (bound+free) plasma concentration, with and without soluble target in plasma, where FIG. 2a shows the curve for dose without soluble target in plasma and FIG. 2b shows the curve for dose with soluble target in plasma;

FIGS. 3a and 3b, show the comparison plots of the results from FIGS. 2a and 2b on the logarithmic scale in FIG. 3a and on the linear scale in FIG. 3b;

FIGS. 4a and 4b show the results of a simulation with a cell count (3×10¹¹) with no internalization for the total (bound+free) plasma concentration, with and without soluble target in plasma, where FIG. 4a shows=the curve for dose without soluble target in plasma and FIG. 4b shows the curve for dose with soluble target in plasma;

FIGS. 5a and 5b show the comparison plots of the results from FIGS. 4a and 4b on the logarithmic scale in FIG. 5a and on the linear scale in FIG. 5b;

FIGS. 6a and 6b show the results of a simulation with a cell count (3×10¹¹) with 100% internalization for the total (bound+free) plasma concentration, with and without soluble target in plasma, where FIG. 6a shows the curve for dose without soluble target in plasma and FIG. 6b shows the curve for dose with soluble target in plasma; and

FIGS. 7a and 7b show the comparison plots of the results from FIGS. 6a and 6b on the logarithmic scale in FIG. 7a and on the linear scale in FIG. 7b.

### DETAILED DESCRIPTION

It has now been found that one or more methods for preparing a patient to eliminate or reduce, and/or modulating dose to account for, existing levels of soluble targets in plasma prior to or during dosing will improve drug performance. For therapeutic drugs, these strategies improve the therapeutic window, that is, maintain tumor cell killing while reducing toxicity to off-target tissues and organs. For diagnostic drugs, these strategies improve visualization of the site of disease.

### Definitions

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used in the specification and claims, the singular form “a”, “an” and “the” include plural references unless the context clearly dictates otherwise.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a combination composition of the invention and/or prodrugs thereof to a subject in need of diagnosis or treatment.

As used herein, the term “ligand”, “targeting vector”, or “targeting ligand” refers to a moiety which is bound to or coordinated to the imaging agents or zwitterionic metal chelators of the combination compositions of the invention to provide enhanced binding to particular cell types or an increased concentration in the presence of particular cell types. In certain embodiments, the targeting vector can be bound to the imaging agents or zwitterionic metal chelators of the combination compositions in addition to the zwitterionic groups thereon. In still other embodiments, the targeting vector can be bound to the zwitterionic metal chelator in place of one or more zwitterionic groups provided that the zwitterionic metal chelator retains at least one zwitterionic group.

As used herein, the term “therapeutic window” or “therapeutic index” refers to the relationship between the therapeutic and toxic dose of a given drug and is calculated using the ED50 and TD50 (Therapeutic Index=TD50/ED50). In certain embodiments of the invention, the zwitterionic metal chelators of the invention have a higher therapeutic index relative to other metal chelators. In certain other embodiments, the therapeutic window refers to a certainty safety factor (CSF) which is defined herein as the ratio of [TD₁/ED₉₉]. A CSF>1 indicates that the dose effective in 99% of the population is less than the dose that would be toxic in 1% of the population. In certain embodiments of the invention, the combination compositions of the invention have a higher CSF relative individual active agents.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

As used herein, the term “combination” refers to a mixture of more than one type of compounds.

As used herein, the term “contacting” refers to the bringing together of substances in physical contact such that the substances can interact with each other. For example, when an agent is “contacted” with tissue or cells, the tissue or cells can interact with the agent, for example, allowing the possibility of binding interactions between the agent and molecular components of the tissue or cells. “Contacting” is meant to include the administration of a substance such as an agent of the invention to an organism. Administration can be, for example, oral or parenteral.

As used herein, the term “ionic group” refers to a moiety comprising one or more charged substituents. The “charged substituent” is a functional group that is generally anionic or cationic when in substantially neutral aqueous conditions (e.g. a pH of about 6.5 to 8.0 or about physiological pH (7.4)). As recited above, examples of charged anionic substituents include anions of inorganic and organic acids such as sulfonate (—SO₃¹⁻), oxide, sulfinate, carboxylate, phosphinate, phosphonate, phosphate, and esters (such as alkyl esters) thereof. In some embodiments, the charged substituent is sulfonate or oxide. Examples of charged cationic substituents include quaternary ammonium ions (—NR₃⁺) and phosphonium ions (—PR₃⁺), where R is independently selected from C₁₋₆ linear alkyl, C₄₋₆ branched alkyl, C₃₋₆ cycloalkyl, aryl, heteroaryl and arylalkyl or heteroarylalkyl. Other charged cationic substituents include protonated primary, secondary, and tertiary amines, and as well as guanidinium or amidinium or pyridinium or other protonated, alkylated or oxygenated nitrogen heterocycles. In some embodiments, the charged substituent is —N(CH₃)₃⁺.

As used herein, the phrase “non-ionic oligomeric or polymeric solubilizing groups” refers to soluble polymers such as, for example, polyethylene glycol, polypropylene glycol, polyethylene oxide and propylene oxide copolymer, a carbohydrate, a dextran, polyacrylamide, a peptide and the like. The solubilizing group can be attached by any desired mode. The point of attachment can be, e.g., a carbon-carbon bond, a carbon-oxygen bond, or a nitrogen-carbon bond. The attachment group can be, e.g., an ester group, a carbonate group, an urea group, an alcohol group, an ether group, a sulfide group, an amino group, an alkylene group, an alkyne group, an azide group, a tetrazine, an amide group, a carbonyl group, or a phosphate group.

Some examples of solubilizing groups include polyethylene glycols, such as —(CH₂CH₂O)ₐ—H, —OC(═O)O(CH₂CH₂O)ₐH, —OC(═O)O(CH₂CH₂O)ₐCH₃, —O(CH₂CH₂O)ₐCH₃, and —S(CH₂CH₂O)₂CH₃, “a” being an integer between about 2 and about 25O. In some embodiments, “a” is 4 to 12 or 5 to 10. In further embodiments, “a” is 6, 7, or 8. Other examples of solubilizing groups include dextrans such as —OC(═O)O(dextran).

The solubilizing moiety can have an absolute molecular weight of from about 500 amu to about 100,000 amu, e.g., from about 1,000 amu to about 50,000 amu or from about 1,500 to about 25,000 amu.

Further examples of solubilizing groups include: —(CH₂): —(OCH₂CH₂)_{d}—OR^{a}, wherein “c” is 0 to 6, “d” is 1 to 200, and R^{a} is H or C₁₋₆ alkyl. In some embodiments, “c” is 1 to 4, “d” is 1 to 10, and R^{a} is H. In some embodiments, “d” is 6 or 7.

See WO 2008/017074, U.S. Ser. No. 12/376,243 (filed Feb. 3, 2009), and U.S. Ser. No. 12/376,225 (filed Feb. 3, 2009), each of which is incorporated herein by reference in its entirety, for a further description of suitable non-ionic oligomeric or polymeric solubilizing groups, and method for incorporating them into dyes.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds of the invention can also include all isotopes of atoms occurring in the intermediates or final compounds. Isotopes include those atoms having the same atomic number but different mass numbers. For example, isotopes of hydrogen include tritium and deuterium.

The chemical substances represented herein by name, chemical formula, or structure are meant to include all stereoisomers, geometric isomers, tautomers, resonance structures, and isotopes of the same, unless otherwise specified.

The chemical substances described herein may be charged or include substituents with formal charges. When such chemical substances are represented as charged, it is understood that, unless otherwise specified, the charges are generally countered with an appropriate counterion. For example, chemical substances or functional groups having a charge of −I are understood to be countered with an ion have a +1 charge. Suitable counterions with +1 charge include Na+, K+, tetraalkylammonium ions, and the like. Conversely chemical substances or functional groups having a charge of +1 are understood to be countered with an ion having a −1 charge. Suitable counterions with −1 charge include F—, Cl—, Br—, I—, perchlorate, acetate, trifluoroacetate, and the like.

As used herein, the term “zwitterionic group,” “zwitterionic ligand,” or “zwitterion” refer to one or more charged moieties with balanced (electrically neutral) polyionicity. The zwitterionic feature could be part of the linker and/or payload and/or targeting ligand. The zwitterionic multimeric drugs of the claimed invention are decorated by one or more zwitterionic groups. In the case of a payload that is a metal chelator, these zwitterionic groups are distinct from metal chelator cores, which typically has negative charges to chelate positively-charged metals. For example, a zwitterionic metal chelator with a chelator core of −4 that binds a +4 metal would have a total net charge of zero. Although a total net charge of zero is considered ideal, a zwitterionic metal chelator with a chelator core of −4 that binds a +2 metal, resulting in an overall charge of −2, would still be expected to exhibit improved properties in vivo because of shielding of the chelator core/metal complex by one or more zwitterionic groups. In the absence of zwitterionic groups, the molecule would have no such shielding or expanded water of hydration and would be more likely to bind non-specifically.

A particular active agent molecule may have several attached “zwitterionic groups” or charge pairs. In general, the anion portion and the cation portion of the zwitterionic group (charge pair) will be part of the same moiety, though it is possible for two ionic groups to be used as separate moieties to form a zwitterionic group. In particular embodiments, the zwitterionic group is covalently bound to the base structure via a carbon-carbon bond, a carbon-oxygen bond, or a nitrogen-carbon bond. Examples of zwitterionic groups (charge pairs) that can be included in the compounds and complexes of the claimed invention include, but are not limited to, ammoniophosphates, ammoniophosphonates, ammoniophosphinates, ammoniosulfonates, ammoniosulfates, ammoniocarboxylates, ammoniosulfonamides, ammonio-sulfon-imides, guanidiniocarboxylates, pyridiniocarboxylates, pyridiniosulfonates, ammonio(alkoxy)dicyanoethenolates, ammonioboronates, sulfoniocarboxylates, phophoniosulfonates, and phosphoniocarboxylates. The charged groups in these zwitterions can be separated by suitable spacer groups like linear or branched alkyl chains, aryl or heteroaryl moieties. In certain embodiments, the zwitterionic groups can be derivatives of amino acids, such as amino carboxylic acids, amino phosphonic acids, amino phosphinic acids or amino sulfonic acids, furthermore, aminoalkyl substituted sulfates or phosphates. Zwitterions can also be derivatives of betaines, such as carboxybetaines, sulfobetaines, sulfabetaines, phosphobetaines or phosphabetaines or N-oxides or derivatives of sulfamic acid. Particular examples of zwitterionic groups include ammonium sulfobetaines or N-oxides. A simple example of a zwitterionic group at physiological pH is the charge pair of a carboxylic acid (deprotonated at physiological pH) and an amine (protonated at physiological pH).

In some embodiments, the targeting ligands of the zwitterionic multimer drugs of the invention can also comprise a targeting vector for an agricultural process, chemical process, disease, or tissue-specific epitope. In certain embodiments, the targeting vector can be dPSMA-617, a small molecule capable of targeting Fibroblast Activation Protein (FAP) also called FAP-inhibitor or FAPI, an amino acid or combination of amino acids, or derivatives thereof.

An ideal active agent conjugated to a targeting vector would adopt the total net charge of the targeting vector, which is purposeful because in most cases the charges on the targeting vector are crucial for the ability to bind its target. Targeted zwitterionic trimer drugs thus retain the major advantage of minimizing non-specific binding while maximizing specific binding. It should be apparent to those skilled in the art that additional charges can be added to the zwitterionic multimer drugs, if needed, to balance overall surface charge to zero.

In certain embodiments, the zwitterionic multimeric drugs comprise a reactive conjugation group. Such reactive conjugation groups are typically an activated derivative of a carboxylic acid, such as an n-hydroxysuccinimide (NHS) ester, a sulfo-NHS ester, a pentafluorophenyl (PFP) ester, a hydroxybenzotriazole (HOBt) ester, a hydroxyazabenzotriazole (HOAt) ester, a tetrafluorophenyl (TFP) ester, an acid anhydride, an acid azide or an acid halide. Such reactive conjugation groups can be bound or substituted onto the chelator at any suitable structural location as would be understood by one of ordinary skill in the synthesis of such compounds. Reactive conjugation groups also include, but are not limited to, alkynes, azides, maleimides, thiols, amines, alkohols, phenols, carbonyls, phosphanes, alkenes and tetrazines.

As used herein, the phrase “core”, “central core”, “branching point” or “nexus” refers to a chemical structure which is located at the center of the drug with four or more substituents for binding zwitterionic linkers and/or targeting ligands. The nexus or “branching” may also be distributed in a more polymer-like configuration.

As used herein, the phrase “linker” refers to a chemical structure connects two or more functional groups.

As used herein, the phrase “payload” refers to a diagnosis or a treatment functional group. When being used as a treatment functional group, the payload refers to, for example, a potent cytotoxin drug that kills cancer cells. Another example would be a payload that radiosensitizes the malignant cell to radiation. When being used as a diagnosis functional group, the payload refers to an imaging agent that provides a signal facilitating the identification of cancer cells.

Other definitions appear in context throughout the disclosure.

### Diagnosis and Treatment of Diseases Complicated by Soluble Target in Blood and Methods for Improving the Same

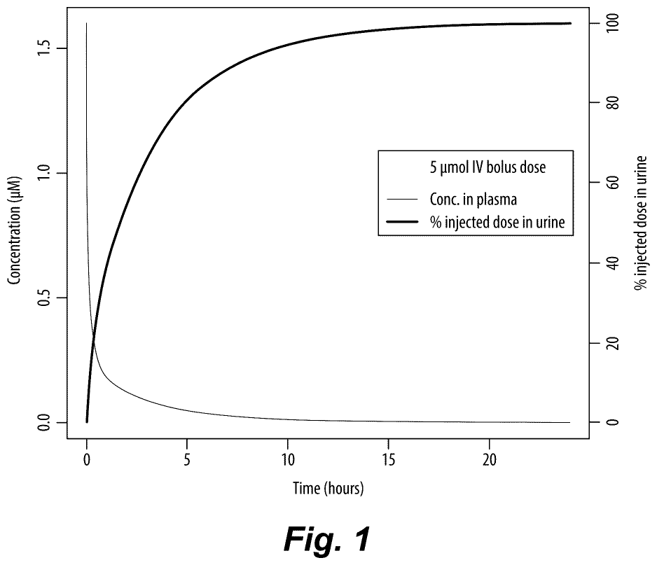

An “ideal” small molecule drug is one that is injected intravenously and rapidly cleared by the kidneys only. Such an ideal molecule is advantageous in many diagnostic and treatment settings. For cancer diagnosis and treatment specifically, an ideal molecule greatly improves cancer cell kill while reducing toxicity. This is because the molecule is disease targeted, but because also clears very quickly through urine, allowing for rapid re-treatment and minimal cancer regrowth between treatments. An “ideal” drug exhibits rapid clearance from plasma and 100% elimination of unbound drug into urine by 24 h. See, for example, FIG. 1.

Additionally, dosing modulation, in this case increasing the concentration of the drug with each subsequent dose is made possible because the concentration of harmful soluble proteins are removed from the body with each dose. While this discussion focuses on FAP and PMSA as the harmful soluble proteins, or the soluble targets, the strategies discussed herein can be readily applied to other disease specific soluble targets as appreciated by one having ordinary skill in the art in view of this disclosure.

Referring now to FIG. 1, FIG. 1 was generated using the standard PBPK model described by Shah et al. A hypothetical small molecule with the following assumptions was injected intravenously, then its blood and urine concentrations and PK parameters were measured over time:
Input Parameters: 4,500 Da (≈1.24 nm hydrodynamic diameter) drug
Bolus (over a few seconds) injection
Renal-only clearance
No other off-target or other binding or association
No renal tubular re-uptake

The results from this analysis shown in FIG. 1 illustrate the following blood and urine curves. The left axis and red curve show plasma concentration over time while the right axis and blue curve show percent of injected dose in urine on linear scale.

However, there presents a problem with soluble targets in plasma when using disease targeted drugs or agents. Soluble targets floating in plasma act as a sink that will sequester the injected drug. The estimated concentration in plasma is discussed in the data below, but in general, the concentration is high enough that the concentration gradient between tumor and plasma should be considered and planned for in determining dosage. Further, since the soluble targets can often be 90 kDa or larger, they cannot be filtered by the kidney, and thus it is not possible to extract the radioactivity from plasma and eliminate it into urine. In certain cases, it may also be possible that a plasma-bound ligand will be recognized by the liver and cleared hepatically.

Certain conventional techniques, such as blocking a soluble target in plasma with cold drug, either by giving extra cold drug or injecting hot drug of low specific activity (which has a high fraction of cold drug in it) has not achieved meaningful improvement. Though these conventional techniques may lower the number of hot atoms bound to plasma, the number of hot atoms bound to malignant cells is proportionally lowered as well, thus achieving no improvement.

### Example: Fibroblast Activation Protein (FAP)

It is known that FAP target concentrations in plasma are high in patients with cancer. Although normal FAP is a 170 kDa transmembrane protein anchored to the surface of fibroblasts, a fraction of the membrane bound protein is cleaved resulting in soluble FAP (sFAP). sFAP is a 97 kDa protein floating in plasma with a relatively long plasma half-life because it is not renally cleared.

For this example, the following assumptions are made for FAP:
Transmembrane FAP=170 kDa
S sFAP or circulating antiplasmin-cleaving enzyme (APCE) is 97 kDa
FAP concentration in plasma is 100 ng/ml (healthy person) up to 500 ng/mL (cancer)
Plasma concentrations and absolute number of molecules in 13 L of plasma:
   100 ng/mL=0.0001 g/L/97,000 g/mole=1 nM
   Equivalent to 1.34×10⁻⁸ mol=8×10¹⁵ molecules in 13 L of plasma
   500 ng/ml=0.0001 g/L/97,000 g/mole=5 nM
   Equivalent to 6.71×10⁻⁸ mol=4×10¹⁶ molecules in 13 L of plasma

Based on these values, at least 100 μg of a 735 Da FAP binding ligand (8.16×1016 molecules) would need to be injected to completely bind soluble FAP in blood with a safety factor of 2. However, the consequence of this is to also block receptors on the cancer-associated fibroblasts that would otherwise be targeted. That is, it improves the tumor-to-background ratio but at the expensive of absolute signal strength.

### Example: Prostate-Specific Membrane Antigen (PMSA)

Like FAP, similar observations have been made in patients with prostate cancer, where serum levels of soluble PSMA. For this example, assuming PMSA=100 kDa, the math provided above with respect to FAP can be readily applied to this example, and the following case:
Age<50, 272.9 ng/ml; age>50, 359.4 ng/ml).
BPH: 117.1 ng/ml
Prostate cancer: 623.1 ng/ml

These values in cancer patients also create the need for high concentrations of cold ligand to block all sites in plasma. However, this has the same negative effect as just described for FAP.

### EXPERIMENTAL

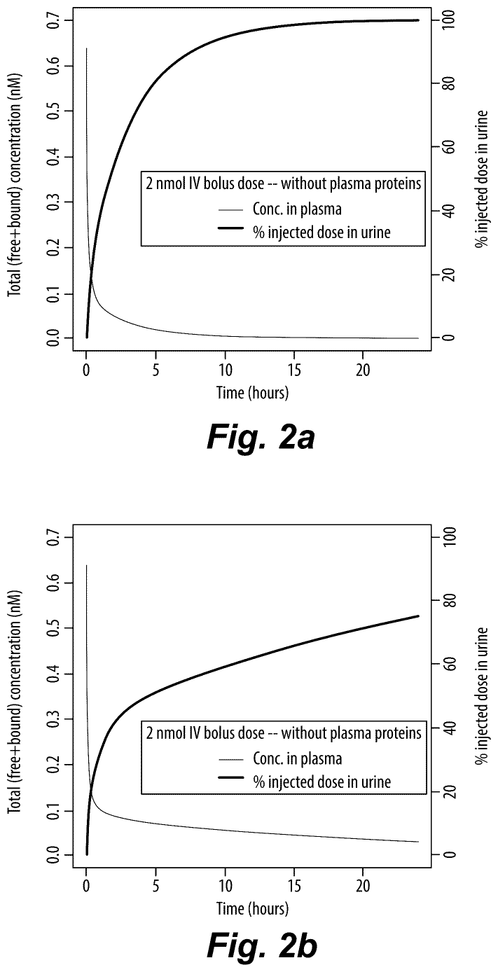

The inventors' experimental computational analysis has shown the effect of 5 nM soluble target in plasma is profound. FIGS. 2a and 2b show the performance of an ideal drug with no soluble target in plasma, and an ideal drug injected into a body with 5 nM of soluble target in plasma. FIG. 2a shows the curve for dose without soluble target in plasma and FIG. 2b shows the curve for dose with soluble target in plasma

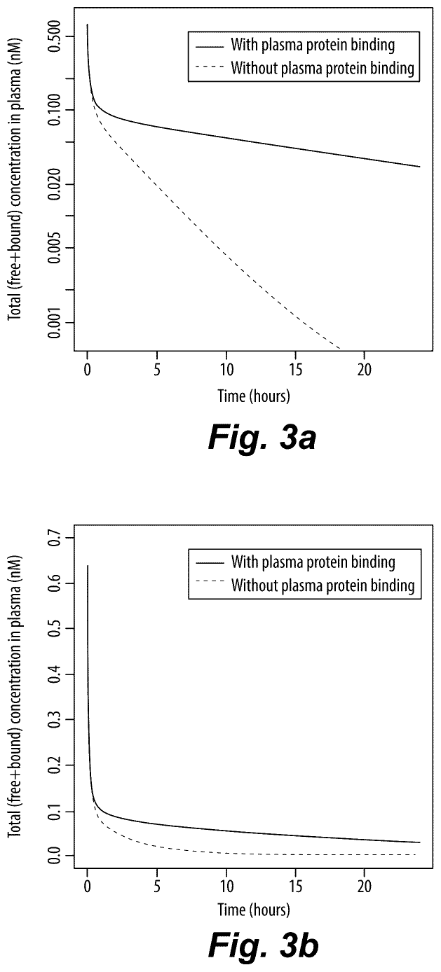

For these experimental simulations, the following parameters were used: a 100 MBq dose was administered, 50 MBq/nmol specific activity, K_{D}=1 nM, Bmax=10⁵, no internalization, and a tumor of 3×10⁸ cells. As shown in FIGS. 2 and 3, a dramatic change in the total drug in plasma curve with the plasma binding as well as dramatic change in the AUC of elimination in urine was observed.

This change can be more easily seen in the comparison plots shown in FIGS. 3a and 3b, for example where FIG. 3a shows the logarithmic scale, and FIG. 3b shows the linear scale.

A summary of AUCs for total (free+bound) in plasma and elimination into urine for the experimental simulation described above is shown below in Table 1.

TABLE 1

| | Plasma AUC | | Plasma AUC | | Urine AUC | Urine AUC |
|---|---|---|---|---|---|---|
| | 0-24 h | | 0-7 d | | 0-24 h | 0-7 d |
| No Plasma Binding | 0.361 | nM h | 0.362 | nM h | 42.1 nmol h | 330.0 nmol h |
| 5 nM Soluble | 1.35 | nM h | 2.14 | nM h | 28.5 nmol h | 300.9 nmol h |
| Target in Plasma | | | | | | |

| | | | | |
|---|---|---|---|---|
| Delta | 3.7 Fold | 5.9 Fold | 32.3% Lower | 8.8% Lower |
| | Higher | Higher | | |

In a second experimental simulation, the same analysis was performed using internalization set to 100% instead of 0%. Virtually the same results as the prior analysis without internalization. This is because the tumor cell count was set very low (3×10⁸), corresponding to a total receptor amount of approximately 0.05 nmol that has a negligible effect on the plasma and urine concentrations. This was repeated again, but without internalization and with 3×10¹¹ cells (1000-fold increase), and then compared to the case with 100% internalization and 3×10¹¹ cells and internalization.

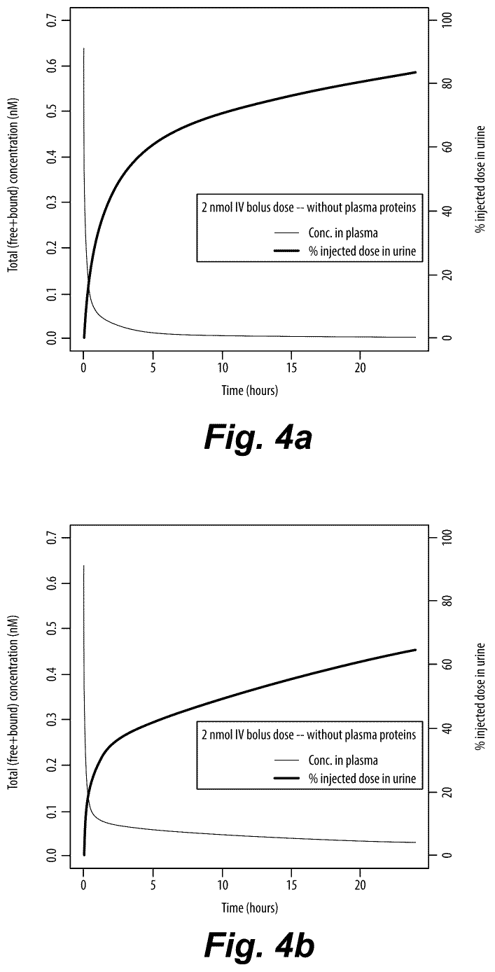

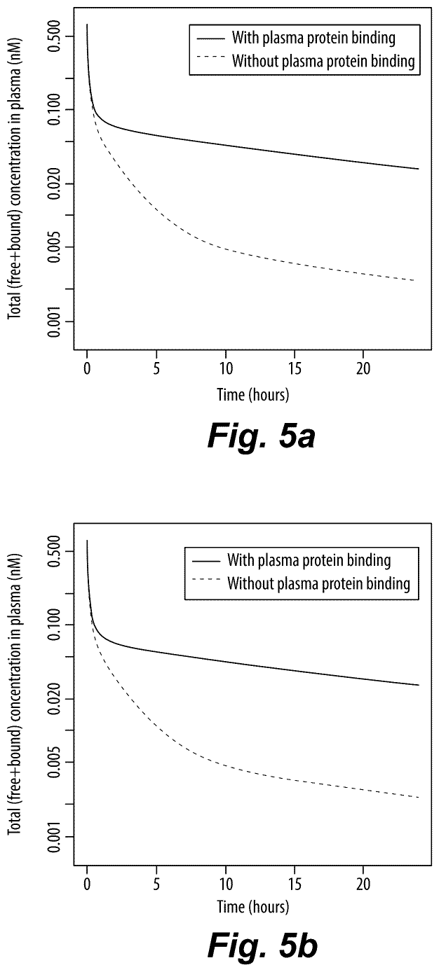

The results for the simulation using the higher cell count (3×10¹¹) and without internalization are shown in FIGS. 4a and 4b, where FIG. 4a show total (bound+free) soluble target in plasma without internalization and where FIG. 4b shows total (bound+free) soluble target in plasma with internalization. The corresponding comparisons between the plasma concentration curves on the logarithmic and linear y-scale are shown in FIGS. 5a and 5b, respectively.

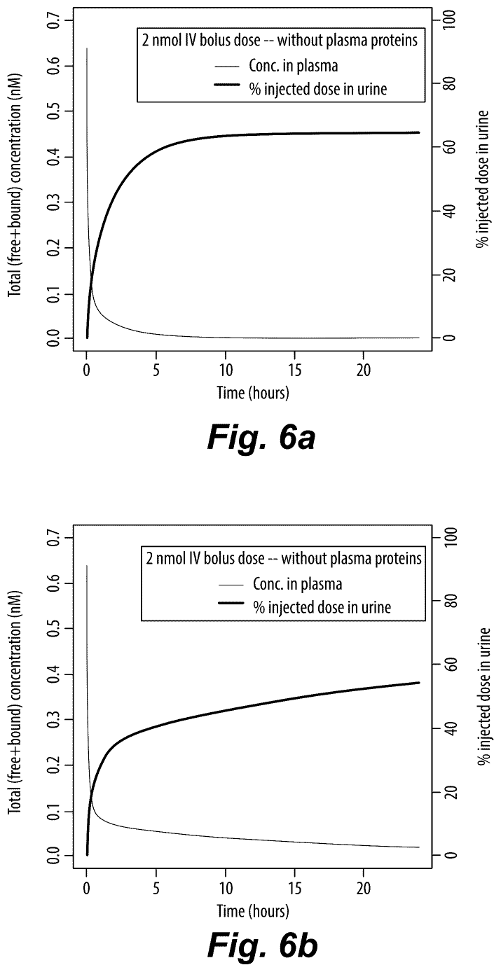

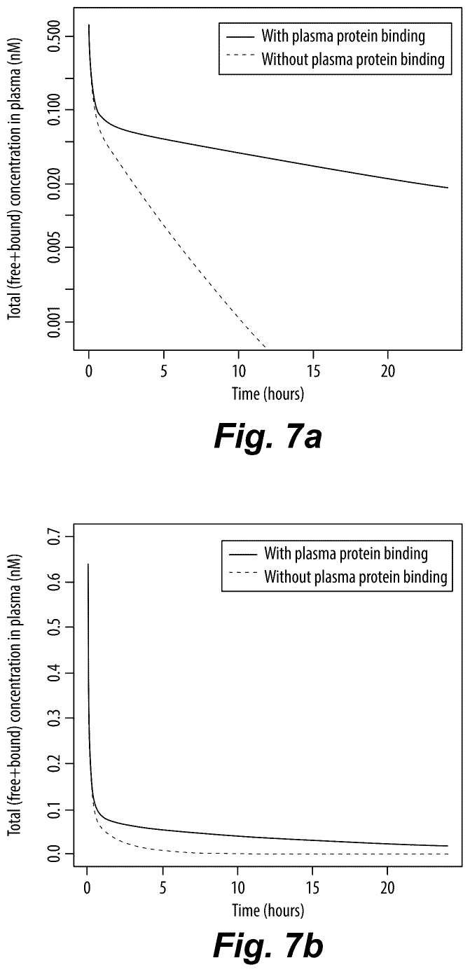

FIGS. 6a and 6b show the results of the simulation repeated with the higher cell count (3×10¹¹) but with internalization (100%) for the total (bound+free) plasma concentration with and without soluble target in plasma. FIG. 6a shows the curve for dose without soluble target in plasma and FIG. 6b shows the curve for dose with soluble target in plasma. The corresponding comparisons between the plasma concentration curves on the logarithmic and linear y-scale are shown in FIGS. 7a and 7b, respectively.

A summary of AUCs for total (free+bound) in plasma and elimination into urine without internalization is shown in Table 2.

TABLE 2

| 3 × 10¹¹ cells without | Plasma AUC | | Plasma AUC | | Urine AUC | Urine AUC |
|---|---|---|---|---|---|---|
| internalization | 0-24 h | | 0-7 d | | 0-24 h | 0-7 d |
| No Plasma Binding | 0.302 | nM h | 0.362 | nM h | 32.8 nmol h | 312.3 nmol h |
| 5 nM Soluble | 1.15 | nM h | 2.13 | nM h | 24.0 nmol h | 283.9 nmol h |
| Target in Plasma | | | | | | |

| | | | | |
|---|---|---|---|---|
| Delta | 3.8-Fold | 5.8-Fold | 26.8% Lower | 9.1% Lower |
| | Higher | Higher | | |

A summary of AUCs for total (free+bound) in plasma and elimination into urine with 3×10¹¹ cells and 100% internalization is shown in Table 3.

TABLE 3

| 3 × 10¹¹ cells with | Plasma AUC | | Plasma AUC | Urine AUC | Urine AUC |
|---|---|---|---|---|---|
| 100% internalization | 0-24 h | | 0-7 d | 0-24 h | 0-7 d |
| No Plasma Binding | 0.23 | nM h | 0.23 nM h | 28.6 nmol h | 214.8 nmol h |
| 5 nM Soluble | 1.0 | nM h | 1.40 nM h | 21.8 nmol h | 202.8 nmol h |
| Target in Plasma | | | | | |

| | | | | |
|---|---|---|---|---|
| Delta | 4.3 Fold | 6.1 Fold | 23.8% Lower | 13% Lower |
| | Higher | Higher | | |

What the inventors have found as a result of these experimental computational analyses is that the overwhelming conclusion is that plasma AUC increases considerably and urine elimination decreases considerably when there is 5 nM soluble target in plasma. This presents a significant issue that needs to be addressed to improve the efficacy of disease targeted diagnostics and treatment. This application therefore provides strategies for addressing this issue.

A comparison was run between no soluble target in plasma, 1 nM (normal levels), and 5 nM (levels in cancer) and examined RBE5 effective radiation to various tissues and organs, as well as the whole body. The input parameters were:
3×10⁸ cell tumor embedded in liver interstitial space
100 MBq dose
50 MBq/nmol specific activity
K_{D}=1 nM
B_{Max}=10⁵
No internalization (worst case)

Outputs were calculated at 48 hours (roughly 4.4 half-lives of Pb-212). The results of the comparison are shown in Table 4.

TABLE 4

| | | | | SUV | | | | | | | | |
|---|---|---|---|---|---|---|---|---|---|---|---|---|
| | Total | RBE5 | RBE5 | (Tumor to | RBE5 | | RBE5 | | RBE5 | | RBE5 | |
| | Cells | Integrated | Integrated | Liver | Integrated | | Integrated | | Integrated | | Integrated | |
| Soluble Target in | (%) | Dose to | Dose to | RBE5 | Dose to | | Dose to | | Dose to Bone | | Total Body | |
| Plasma | Killed * | Tumor | Liver | Ratio) | Kidney | | Lymph Nds | | Marrow | | Dose | |
| 0 (Ideal Drug) | 1705 | 15,847 mSv | 80.6 mSv | 196.6 | 48.9 | mSv | 217 | mSv | 58.1 | mSv | 62.0 | mSv |
| 1 nM (Normal) | 1567 | 14,563 mSv | 118.4 mSv | 122.9 | 72.2 | mSv | 314.1 | mSv | 84.8 | mSv | 88.9 | mSv |
| % Change | −8% | −8% | +47% | −37% | +48% | | +48% | | +48% | | +43% | |
| 5 nM | 1199 | 11,148 mSv | 226.9 mSv | 49.1 | 139.5 | mSv | 566.2 | mSv | 158.4 | mSv | 158.5 | mSv |

| | | | | | | | | |
|---|---|---|---|---|---|---|---|---|
| % Change | −30% | −30% | +2.8-Fold | −4-Fold | +2.9-fold | +2.6-Fold | +2.7-Fold | +2.6-Fold |

* Cells Killed via Bystander effect (%) = RBE5 Integrated Dose to Tumor * Kbvstander/3 Sv * 100%.

### Results

The results of this study provide important and unexpected findings, summarized in this section.

First, in the presence of 5 nM soluble target in plasma, at 24 h there is a 4-fold increase in plasma AUC and a 32% decrease in urine elimination. Although the difference in urine elimination is reduced to 9% by day 7, an ideal situation provides the highest possible 24 h clearance in order to achieve the lowest off-target radiation dose. Second, the total cancer cells killed with 5 nM soluble target in plasma vs. without differs by 30%. Third, the impact of soluble target in plasma will be greatest at the first dose. The highest concentration of FAP and PSMA in plasma is in the setting of high cancer burden. Reducing cancer burden should return plasma levels to baseline, which are ≈1 nM where the impact of soluble target is still present but much less. Fourth, liver dose (and the dose to many other organs) increase 2.6 to 2.8 fold in the presence of 5 nM soluble target in plasma, due to higher concentration and perfusion. Combining this with the lower tumor dose results in 4-fold reduction in SUV. Fifth, the concentration of drug in liver (vascular+interstitial) is similar to bone marrow but the ECF to whole organ volume ratio of marrow is (67.2+567)/3050=0.2. Therefore, the final dose in the liver should be around 1.4-fold higher than in marrow, which is shown to be found in Table 4. Sixth, lymph node dose is 3.5-fold higher than bone marrow dose.

The results support the inventor's proposed novel and inventive methods for improving the outcome treatment and diagnostic procedures by removing certain soluble targets in a patient's plasma before administering a disease targeted diagnostic or treatment to the patient.

### Discussion

The following discussion provides two strategies, which can be used individually or in combination, to prepare a patient for receiving a disease targeted diagnostic agent or treatment agent so that said agent is able to perform more like an “ideal” small molecule drug. The first strategy is extracorporeal treatment of plasma to remove a soluble target from the blood, and the second strategy is using dose modulation to proportionally increase the concertation of the diagnostic/treatment agent relative to the concentration of the soluble target in the blood. Each strategy will be discussed in turn.

Strategy 1: Extracorporeal Removal of Soluble Target from Blood

The removal of the soluble target from the blood can be performed using two different methods, method A and method B.

The first method, method A, is plasmapheresis, using plasma exchange. Here, the patient's plasma is removed and discarded while fresh plasma (e.g., from the blood bank) is infused. This step non-specifically reduces all cancer-dependent increases in soluble target and returns soluble target to normal levels seen in non-cancer patients. The advantage of this “open loop” method is that that plasmapheresis is widely available and all cancer-associated soluble targets that are elevated in plasma are removed because the patient's plasma is discarded and the patient is infused with fresh plasma pooled from normal donors. A potential disadvantage of method A is that levels of the soluble target are only brought to “normal levels,” i.e. those seen in the general population, which may still be too high for ideal drug pharmacokinetics. In such a case, it is possible to combine method A with method B, as will be described below, to overcome this disadvantage.

The second method, method B, is immunoadsorption. Immunoadsorption is a closed loop method where the patient's plasma is separated from blood and passed over an adsorption column to which a targeting ligand(s) are bound. The soluble target in the plasma attaches to the column and is removed from the patient's plasma, which is then re-infused into the patient. After a complete cycling of the patient's plasma, nearly 100% of soluble target should be removed.

One or both of these two methods is administered before the patient receives a dose of the diagnostic or treatment agent to improve the efficacy of the diagnostic or treatment agent by allowing higher dosing. Since the concentration of the plasma target is significantly reduced or eliminated, there is less risk to off-target exposure of drug, which in the case of radioactive drugs means a lower risk of complications from radiation damage. Thus, using the described preparation methods to prepare the patient for receiving the diagnostic or treatment agent allows a maximum dose of the agent to be administered as the first dose.

In certain embodiments, the soluble target identified in the plasma can be fibroblast activation protein (FAP) and/or prostate-specific membrane antigen (PMSA). Accordingly, the filter medium in the immunoadsorption column can include a ligand(s) that binds these targets. Specific embodiments of said drugs and their specific structures are described in further detail in U.S. patent application Ser. No. 19/209,293, filed May 15, 2025, U.S. Provisional Patent Application No. 63/858,228, filed Aug. 5, 2025, and U.S. Provisional Paten Application No. 63/902,968, filed Oct. 21, 2025, each of which is incorporated herein by reference in its entirety, as well as discussed herein below.

This first strategy, i.e., either method A or method B, can also be used in a method of improving diagnosis and treatment of diseases complicated by soluble target in blood, by improving the results of such diagnostics or treatment, by: i) increasing tumor-to-background ratio during diagnostic imaging; ii) by allowing the patient to rapidly clear the agent so that the agent can be rapidly re-dosed (e.g., time between doses is decreased); and iii) by allowing the first dose to be a higher dose than for an unprepared patient.

In certain embodiments, the method of improving can comprise the steps of a) preparing a patient using method A or method Bas discussed above, and b) administering a disease targeted diagnostic agent or treatment agent to the patient at a higher dose than if the patient had not been prepared using the method of preparing the patient. In certain embodiments, steps a) and b) are performed sequentially.

Similarly, in certain embodiments, a method of improving diagnosis and treatment of diseases complicated by soluble target in blood can comprise the sequential steps of, preparing a patient using either method A or method B described above, and administering a disease targeted diagnostic agent or treatment agent to the patient such that at least 80% of an injected dose of the disease targeted diagnostic agent or treatment agent not binding the target is passed through urine within 48 hours of administration. In certain embodiments, at least 90% of the injected dose of the disease targeted diagnostic agent or treatment agent not binding the target is passed through urine within 48 hours of administration. In certain embodiments, at least 80% of the injected dose of the disease targeted diagnostic agent or treatment agent not binding the target is passed through urine within 24 hours of administration. In certain embodiments, at least 90% of an injected dose of the disease targeted diagnostic agent or treatment agent not binding the target is passed through urine within 24 hours of administration.

Similarly, in certain embodiments, a method of improving diagnosis and treatment of diseases complicated by soluble target in blood can comprise the sequential steps of preparing a patient using either method A or method B described above, and administering the disease targeted diagnostic agent or treatment agent to the patient such that a prescribed concentration of an injected dose of the disease targeted diagnostic agent or treatment agent is removed from blood faster, is passed through urine within a prescribed time after administration, where the prescribed concentration is greater than if the patient had not been prepared, and where the prescribed time after administration is less than if the patient had not been prepared.

### Strategy 2: Dose Modulation

For this strategy, the dose of the diagnostic or treatment agent is modulated from the first dose and for each subsequent dose to account for changes in soluble target in plasma due to each preceding dose. For example, assuming a dose “X” for dose 1 and a soluble target in plasma concentration of “Y”. If “Y” is reduced by 5-fold after the first dose because treating the disease will also cause the soluble target in plasma to be reduced, then “X” can be increased by 5-fold in the second dose, and the patient will receive the same off-target dose. This is particularly important in treatments involving targeted radiation. Using this method, the agent dose “X” will be adjusted based on the change in soluble concentration “Y.” In certain cases, it may be that two doses of “X” before “Y” lowers and then “X” is adjusted appropriately for the next dose. This method will guarantee that the patient is always receiving the maximum possible dose without overdosing off-target normal tissues and organs.

In certain cases, not may not be necessary to take any preparation steps to lower the initial concentration of the soluble target in plasma. Instead, by giving a lower dose at the first dose and at the highest level tolerated due to off-target dose, subsequent doses can be adjusted accordingly to increase proportional to the fall of soluble target as the patient is treated. This can be due to a decrease in tumor size, if the patient is being treated for cancer, for example. Accordingly, it is possible to achieve the improved outcomes of strategy 2, without also employing strategy 1.

Strategy 2 can be used for improving diagnosis and treatment of diseases complicated by soluble target in blood. In certain embodiments, this method can include the steps of a) administering a first dose of a disease targeted diagnostic agent or treatment agent to the patient at a first concentration, wherein the disease targeted diagnostic agent or treatment agent is targeted at a biological abnormality caused by a disease overexpressing the soluble target such that the first dose lowers an initial concertation of the soluble target in the plasma of the patient, and b) administering additional subsequent doses of the disease targeted diagnostic agent or treatment agent to the patient at a respective subsequent concentration that increases with each administration at a rate proportional to the decrease of the concentration of the soluble target in the plasma of the patient.

In certain embodiments, administering additional subsequent doses can include administering the additional subsequent doses at a maximum concentration without overdosing off-target, non-diseased tissues and organs.

In certain embodiments, the disease targeted diagnostic agent or treatment agent can be or include a small-zwitterionic molecule having a core chemical structure located at a center and two or more substituents for binding zwitterionic linkers and a targeting ligand. Specific embodiments of said drugs and their specific structures are described in further detail in U.S. patent application Ser. No. 19/209,293, filed 15, 2025. In certain embodiments, the soluble target is or includes fibroblast activation protein (FAP) and/or prostate-specific membrane antigen (PMSA), and the targeting ligand is a molecule that binds FAP or PMSA

In certain embodiments, administering additional subsequent doses includes administering the additional subsequent doses such that at least 80% of an injected dose of the disease targeted diagnostic agent or treatment agent is passed through urine within 48 hours of administration. In certain embodiments, at least 90% of the injected dose the disease targeted diagnostic agent or treatment agent is passed through urine within 48 hours of administration. In certain embodiments, at least 80% of the injected dose the disease targeted diagnostic agent or treatment agent is passed through urine within 24 hours of administration. In certain embodiments, at least 90% of the injected dose the disease targeted diagnostic agent or treatment agent is passed through urine within 24 hours of administration.

Strategy 3: Extracorporeal Removal of Soluble Target from Blood Combined with Dose Modulation

In certain embodiments, it may be desired to combine strategies 1 and 2 together. In this case, the method of preparing a patient can include, a) preparing the patient as described above (either method A or method B of strategy 1), b) administering a first dose of a disease targeted diagnostic agent or treatment agent to the patient at a first concentration, wherein the disease targeted diagnostic agent or treatment agent is targeted at a biological abnormality caused by a disease overexpressing the soluble target such that the first dose lowers an initial concertation of the soluble target in the plasma of the patient, and c) administering a subsequent dose of the disease targeted diagnostic agent or treatment agent to the patient at a second concentration higher than the first concentration, wherein the second concentration is determined as a function of a decrease in the initial concentration of the soluble target in the plasma.

In certain embodiments, the step c) includes administering additional subsequent doses of the disease targeted diagnostic agent or treatment agent to the patient at a respective subsequent concentration that increases with each administration at a rate proportional to the decrease of the concentration of the soluble target in the plasma of the patient.

### Small Zwitterionic Multimeric Targeted Drugs

In certain embodiments, the structure of the small zwitterionic multimeric targeted anti-cancer drug includes a nexus, more than one targeting ligands, linkers, a payload for imaging or treating the tumors, and optionally spacing groups. FIG. 1 shows a generalized structure of the small zwitterionic multimeric targeted anti-cancer drug, particularly a small zwitterionic trimeric targeted drug with three targeting ligands. A forcefield of hydration and balanced charge can be formed around the nexus and linkers conjugating the targeting ligands to the nexus, while the targeting ligands are exposed out of the forcefield such that they can bind to targets on cancer cells or cancer-associated tissue. Specific embodiments of said drugs and their specific structures are described in further detail in U.S. patent application Ser. No. 19/209,293, filed May 15, 2025, U.S. Provisional Patent Application No. 63/858,228, filed Aug. 5, 2025, and U.S. Provisional Paten Application No. 63/902,968, filed Oct. 21, 2025, each of which is incorporated herein by reference in its entirety.

In certain embodiments, a small zwitterionic multimeric targeted drug includes a nexus having multiple attaching sites, multiple targeting ligands conjugated to the nexus via the multiple attaching sites, one or more payloads of a zwitterionic diagnostic or therapeutic agent conjugated to the nexus via one of the multiple attaching sites, and zwitterionic flexible linkers connecting each of the multiple different targeting ligands and the payload to the nexus. In certain embodiments, in addition to the zwitterionic flexible linkers, one or more spacing groups e.g. Ahx locate between a targeting ligand and the nexus.

The central core, the payload of the zwitterionic diagnostic or therapeutic agent, and zwitterionic flexible linkers of the small zwitterionic multimeric targeted anti-cancer drug can form a “force field” of charge-balanced polyionicity and water of hydration such that only the targeting ligands are exposed out of the force field.

These small zwitterionic trimeric targeted drugs and anti-cancer drugs have significant advantages over traditional Antibody-Drug Conjugates (ADCs). First, with respect to the pharmacokinetic profile, the small zwitterionic multimeric targeted anti-cancer drugs of the present invention have a rapid effect and can be applied in a form of multiple re-treatment to prevent resistance to the drugs. Moreover, the small zwitterionic multimeric targeted anti-cancer drugs as described herein are heterotrimeric monovalent molecules which addresses the tumor heterogeneity more efficiently. In addition, the small zwitterionic multimeric targeted anti-cancer drugs de-scribed herein can be synthesized more easily and at a lower cost than ADCs. The small zwitterionic multimeric targeted anti-cancer drug can effectively address tumor cell heterogeneity and off-target/non-specific binding and uptake. The small zwitterionic multimeric targeted anti-cancer drug can include a nexus, a plurality of different targeting ligands conjugated to the nexus, a payload of a zwitterionic diagnostic or therapeutic agent, and zwitterionic flexible linkers connecting each of the different targeting ligands and the payload to the nexus.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

1. A method for preparing a patient for a diagnostic procedure and/or for receiving a therapy using a disease targeted drug, comprising the steps of:
a) identifying one or more soluble targets detectable in plasma of the patient; and
b) extracorporeally removing the soluble targets from the plasma of the patient.

2. The method of claim 1, wherein extracorporeally removing the soluble targets from the plasma of the patient in step b) includes performing plasmapheresis, and wherein performing plasmapheresis further includes:
c) performing plasma exchange, including removing blood from the patient and separating plasma from the blood, discarding the plasma, and providing new plasma to the patient to non-selectively remove the one or more soluble targets within the plasma of the patient.

3. The method of claim 2, wherein the soluble target is fibroblast activation protein (FAP) and/or prostate-specific membrane antigen (PMSA).

4. The method of claim 2, further comprising the steps of:
d) administering a first dose of a disease targeted diagnostic agent or treatment agent to the patient at a first concentration, wherein the disease targeted diagnostic agent or treatment agent is targeted at a biological abnormality caused by a disease overexpressing the one or more soluble targets such that the first dose lowers an initial concertation of the one or more soluble targets in the plasma of the patient; and
e) administering a subsequent dose of the disease targeted diagnostic agent or treatment agent to the patient at a second concentration higher than the first concentration, wherein the second concentration is determined as a function of a decrease in the initial concentration of the one or more soluble targets in the plasma.

5. The method of claim 2, further comprising the steps of:
d) administering a first dose of a disease targeted diagnostic agent or treatment agent to the patient at a first concentration, wherein the disease targeted diagnostic agent or treatment agent is targeted at a biological abnormality caused by a disease overexpressing the one or more soluble targets such that the first dose lowers an initial concertation of the one or more soluble targets in the plasma of the patient; and
e) administering additional subsequent doses of the disease targeted diagnostic agent or treatment agent to the patient at a respective subsequent concentration that increases with each administration at a rate proportional to a decrease of the concentration of the one or more soluble targets in the plasma of the patient.

6. The method of claim 1, wherein extracorporeally removing the soluble targets from the plasma of the patient in step b) includes extracorporeally removing the one or more soluble targets from the plasma of the patient via immunoadsorption.

7. The method of claim 6, wherein immunoadsorption further includes:
c) removing blood from the patient and separating plasma from the blood;
d) passing the plasma through an immunoadsorption column to selectively remove the soluble target identified in step a) from the plasma; and
e) returning the treated plasma to the patient.

8. The method of claim 7, wherein the immunoadsorption column includes a filter medium having one or more targeting ligands bound thereto.

9. The method of claim 8, wherein the one or more soluble targets is fibroblast activation protein (FAP) and/or prostate-specific membrane antigen (PMSA).

10. The method of claim 9, wherein the one or more targeting ligands bound to the filter medium bind FAP or PMSA.

11. The method of claim 6, further comprising the steps of:
f) administering a first dose of a disease targeted diagnostic agent or treatment agent to the patient at a first concentration, wherein the disease targeted diagnostic agent or treatment agent is targeted at a biological abnormality caused by a disease overexpressing the one or more soluble targets such that the first dose lowers an initial concertation of the one or more soluble targets in the plasma of the patient; and
g) administering a subsequent dose of the disease targeted diagnostic agent or treatment agent to the patient at a second concentration higher than the first concentration, wherein the second concentration is determined as a function of a decrease in the initial concentration of the one or more soluble targets in the plasma.

12. The method of claim 6, further comprising the steps of:
f) administering a first dose of a disease targeted diagnostic agent or treatment agent to the patient at a first concentration, wherein the disease targeted diagnostic agent or treatment agent is targeted at a biological abnormality caused by a disease overexpressing the one or more soluble targets such that the first dose lowers an initial concertation of the one or more soluble targets in the plasma of the patient; and
g) administering additional subsequent doses of the disease targeted diagnostic agent or treatment agent to the patient at a respective subsequent concentration that increases with each administration at a rate proportional to the decrease of the concentration of the one or more soluble targets in the plasma of the patient.

13. A method of improving diagnosis and treatment of diseases complicated by one or more soluble targets in blood, comprising the steps of:
a) preparing a patient using the method of claim 1; and
b) administering a disease targeted diagnostic agent or treatment agent to the patient at a higher dose than if the patient had not been prepared using the method of claim 1.

14. The method of claim 13, wherein the steps a) and b) are performed sequentially,
and wherein the step b) further comprises administering the disease targeted diagnostic agent or treatment agent to the patient such that at least 80% of an injected dose of the disease targeted diagnostic agent or treatment agent not binding the target is passed through urine within 48 hours of administration.

15. The method of claim 14, wherein at least 90% of the injected dose of the disease targeted diagnostic agent or treatment agent not binding the target is passed through urine within 48 hours of administration.

16. The method of claim 14, wherein at least 80% of the injected dose of the disease targeted diagnostic agent or treatment agent not binding the target is passed through urine within 24 hours of administration.

17. The method of claim 16, wherein at least 90% of the injected dose of the disease targeted diagnostic agent or treatment agent not binding the target is passed through urine within 24 hours of administration.

18. (canceled)

19. A method of improving diagnosis and treatment of diseases complicated by soluble target in blood, comprising the steps of:
a) administering a first dose of a disease targeted diagnostic agent or treatment agent to the patient at a first concentration, wherein the disease targeted diagnostic agent or treatment agent is targeted at a biological abnormality caused by a disease overexpressing the soluble target such that the first dose lowers an initial concertation of the soluble target in the plasma of the patient; and
b) administering additional subsequent doses of the disease targeted diagnostic agent or treatment agent to the patient at a respective subsequent concentration that increases with each administration at a rate proportional to the decrease of the concentration of the soluble target in the plasma of the patient.

20. (canceled)

21. The method of claim 19, wherein the disease targeted diagnostic agent or treatment agent includes a small-zwitterionic molecule having a core chemical structure located at a center and two or more substituents for binding zwitterionic linkers and a targeting ligand bound to the core.

22. The method of claim 19, wherein the soluble target is fibroblast activation protein (FAP) and/or prostate-specific membrane antigen (PMSA), and wherein the targeting ligand is a molecule that binds FAP or PMSA.

23. The method of claim 19, wherein administering additional subsequent doses includes administering the additional subsequent doses such that at least 80% to 90% of an injected dose of the disease targeted diagnostic agent or treatment agent not binding the target is passed through urine within 24 to 48 hours of administration.
